SEQUENCE LISTING (6 sequences)

SEQ ID NO: 1 - DNA, 355 bp, synthetic construct
  source 1..355: mol_type = other DNA
agtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagt

SEQ ID NO: 2 - DNA, 245 bp, synthetic construct
  source 1..245: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtga

SEQ ID NO: 3 - DNA, 945 bp, synthetic construct
  source 1..945: mol_type = other DNA
agaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccattt

SEQ ID NO: 4 - protein, 73 aa, synthetic construct
MGSWSEFWARLGAIRERLDALGGSEAELAAFEKEIAAFESELQAYKGKGNPEVEKLRYTAGTIKRFLQAYRHN

SEQ ID NO: 5 - protein, 307 aa, synthetic construct
MGSWSEFWARLGAIRERLDALGGSEAELAAFEKEIAAFESELQAYKGKGNPEVEKLRYTAGTIKRFLQAYRHNGGGGDGGGGSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 216 bp, synthetic construct
  source 1..216: mol_type = other DNA
ggttcttgggttttctcgcaacagcaggttctgcaatgggcgcggcgtccctgacgctgtcggctcagtcccggactttactggccgggatagtgcagcaacagcaacagctgttggacgtggtcaagagacaacaagaactgttgcgactgaccgtctggggaacgaaaaacctccaggcaagagtcactgctatagagaagtacctacaggacc

(19) United States
(12) Patent Application Publication
(10) Pub. No.: US 2026/0108629 A1
(43) Pub. Date: Apr. 23, 2026

(54) VECTORS FOR PRODUCTION OF THERAPEUTIC CONSTRUCTS

(71) Applicant: Kite Pharma, Inc., Santa Monica, CA (US)

(72) Inventor: Edward Liao, Petaluma, CA (US)

(21) Appl. No.: 19/298,435

(22) Filed: Aug. 13, 2025

Related U.S. Application Data
(60) Provisional application No. 63/683,157, filed on Aug. 14, 2024.

(51) Int. Cl. A61K 48/00; A61K 40/31; A61K 40/42; C07K 14/47; C07K 14/705; C07K 14/725; C07K 16/28; C12N 15/86

(52) U.S. Cl. CPC A61K 48/005; A61K 40/31; A61K 40/4215; C07K 14/4705; C07K 14/7051; C07K 14/70521; C07K 14/70578; C07K 16/2878; C12N 15/86; A61K 2239/13; A61K 2239/21; C07K 2319/02; C12N 2740/15043; C12N 2740/15051; C12N 2830/48

(57) ABSTRACT

The present disclosure provides DNA molecules, such as transfer plasmids, useful for manufacturing packaged retroviral vectors encoding a therapeutic protein such as chimeric antigen receptors (CARs) and T cell receptors (TCRs). The encoded retroviral transcript in the DNA molecule includes a transgene, an elongation factor 1-alpha 1 (EF1α) promoter, without the EF1α intron, operably linked to the transgene and a Rev response element (RRE).

### CROSS REFERENCE TO RELATED APPLICATIONS

This application claims the benefit under 35 U.S.C. § 119 (e) of the U.S. Provisional Application Ser. No. 63/683,157 filed Aug. 14, 2024, the content of which is hereby incorporated by reference in its entirety.

### REFERENCE TO AN ELECTRONIC SEQUENCE LISTING

The content of the electronic sequence listing (K-1168.xml; Size: 8,300 bytes; and Date of Creation: Jul. 10, 2025) is herein incorporated by reference in its entirety.

### BACKGROUND

Gene and cell therapies represent a frontier in medical treatment, offering hope for the cure of many genetic and acquired diseases by directly addressing their genetic underpinnings. These therapies often rely on vectors to deliver therapeutic genes into target cells. Vectors are delivery vehicles engineered to introduce therapeutic genes into cells, enabling the expression of proteins that can correct or ameliorate disease processes.

Vectors can be viral or non-viral. Viral vectors use the natural ability of a virus to insert its genetic material into a host cell. By replacing the virus's harmful genetic information with therapeutic genes, scientists can harness this mechanism to treat diseases at their genetic roots. Common viral vectors include adenoviruses, gammaretroviruses, lentiviruses, and adeno-associated viruses (AAV), each with its own set of advantages and limitations in terms of capacity, target cell specificity, and immune response elicitation.

The choice of vector depends on various factors including the type of disease, the target cell, the need for temporary or permanent gene expression, and the potential for immune response. There remains a significant, unmet need for the development of safer and more efficient vectors.

Lentivirus, a subset of retroviruses, has garnered significant attention. Characterized by their ability to integrate genetic material into the host cell's DNA, lentiviruses offer a stable and long-lasting method of gene delivery, making them an invaluable tool in the development of gene therapies.

Lentiviruses have a broad range of target cells, including non-dividing cells, which expands their applicability in gene therapy. Their versatility and efficiency in gene delivery have led to their use in various therapeutic interventions, including the modification of immune cells to fight cancer, the correction of genetic defects in inherited disorders, and the delivery of genes to induce the regeneration of damaged tissues.

In the realm of cancer treatment, for example, lentiviral vectors are used to engineer T cells to express chimeric antigen receptors (CARs) or T cell receptors (TCRs), thereby enhancing their ability to recognize and kill cancer cells. A major challenge in the preparation of CAR or TCR cells is the preparation of packaged viral vectors with the coding sequences and the transfer of the coding sequences to a target immune cell.

### SUMMARY

The present disclosure, in various embodiments, provides DNA molecules, such as transfer plasmids, useful for manufacturing packaged retroviral vectors encoding a therapeutic protein such as chimeric antigen receptors (CARs) and T cell receptors (TCRs). The encoded retroviral transcript in the DNA molecule includes a transgene, an elongation factor 1-alpha (EF1α) promoter operably linked to the transgene and a Rev response element (RRE). In one embodiment, the EF1α promoter does not include an EF1α intron. Such a DNA molecule, as demonstrated in the Examples, achieved high efficiency of retroviral vector production, nuclear export, and packaging in the cytoplasm of the host cell.

In accordance with one embodiment of the present disclosure, provided is a DNA molecule encoding a retroviral transcript, wherein the retroviral transcript comprises: (a) a transgene; (b) an elongation factor 1-alpha 1 (EF1α) promoter operably linked to the transgene; (c) two long terminal repeats (LTR) flanking the EF1α promoter and the transgene; and (d) a Rev response element (RRE), wherein the DNA molecule further comprises a viral promoter for driving the transcription of the retroviral transcript, and wherein the EF1α promoter does not include an EF1α intron. In some embodiments, the retroviral transcript does not include an EF1α intron.

In some embodiments, the retroviral transcript is a lentiviral transcript. In some embodiments, the viral promoter is a Cytomegalovirus (CMV) promoter, a SV40 promoter or a Rous Sarcoma Virus (RSV) promoter.

Another embodiment of the present disclosure provides DNA molecule which, when transcribed in a eukaryotic cell, generates retroviral transcript, wherein the retroviral transcript comprises (a) a transgene; (b) an elongation factor 1-alpha 1 (EF1α) promoter operably linked to the transgene; (c) two long terminal repeats (LTR) flanking the EF1α promoter and the transgene; and (d) a Rev response element (RRE), wherein the EF1α promoter does not include an EF1α intron. In some embodiments, the retroviral transcript does not include an EF1α intron.

Yet another embodiment provides a DNA molecule which, when transcribed in a eukaryotic cell, generates a plurality of retroviral transcripts, wherein each retroviral transcript comprises (a) a transgene; and (b) an elongation factor 1-alpha 1 (EF1α) promoter operably linked to the transgene. In some embodiments, less than 80% of the retroviral transcripts further comprise an EF1α intron. In some embodiments, less than 50% of the retroviral transcripts comprise an EF1α intron. In some embodiments, less than 10% of the retroviral transcripts comprise an EF1α intron. In some embodiments, none of the retroviral transcripts comprise an EF1α intron.

In some embodiments, the eukaryotic cell is a mammalian cell. In some embodiments, the eukaryotic cell is a human cell.

In some embodiments, each retroviral transcript further comprises: (c) two long terminal repeats (LTR) flanking the EF1α promoter and the transgene; and (d) a Rev response element (RRE).

In some embodiments, the RRE is upstream to the EF1α promoter or downstream to the transgene. In some embodiments, the RRE is a human immunodeficiency viruses (HIV) RRE. In some embodiments, the RRE is an RRE of an HIV subtype selected from the group of subtypes A1, A2, B, C, D, F1, F2, G and H. In some embodiments, the RRE comprises a nucleic acid sequence having at least 90% sequence identity to SEQ ID NO:1.

In some embodiments, the EF1α promoter comprises a nucleic acid sequence having at least 90% sequence identity to SEQ ID NO:2. In some embodiments, the intron of the EF1α promoter comprises a nucleic acid having at least 90% sequence identity to SEQ ID NO:3.

In some embodiments, the transgene is a therapeutic protein. In some embodiments, the therapeutic protein comprises chimeric antigen receptor (CAR). In some embodiments, the CAR comprises an antigen-binding peptide. In some embodiments, the antigen-binding peptide has binding specificity to the B-cell maturation antigen (BCMA). In some embodiments, the antigen-binding peptide comprises an amino acid sequence having at least 80% sequence identity to SEQ ID NO: 4. In some embodiments, the antigen-binding peptide comprises an amino acid sequence having at least 90% sequence identity to SEQ ID NO:4. In some embodiments, the CAR further comprises one or more of a signal peptide, a transmembrane domain, a costimulatory domain from 4-1BB, CD28, ICOS(CD278), OX40(CD134), or DAP-10, and a CD35 stimulatory domain. In some embodiments, the CAR comprise an amino acid sequence having at least 90% sequence identity to SEQ ID NO:4.

In some embodiments, the retroviral transcript further comprises a posttranscriptional regulatory element (PRE). In some embodiments, the PRE is a Woodchuck PRE (WPRE).

In some embodiments, the DNA molecule is a plasmid.

Also provided, in one embodiment is a retroviral transcript encoded by the DNA molecule of any preceding claim. Still further provided, one embodiment is an in vitro or in vivo cell comprising the DNA molecule of the instant disclosure.

Further provided, in one embodiment, is a method for preparing a retroviral transcript, comprising introducing a DNA molecule of the present disclosure to a eukaryotic cell and culturing the cell to allow the DNA molecule to transcribe to generate the retroviral transcript.

Yet another embodiment provides a retroviral transcript which is prepared by introducing a DNA molecule of the present disclosure to a eukaryotic cell and culturing the cell to allow the DNA molecule to transcribe to generate the retroviral transcript. In some embodiments, the method further comprises collecting a virus packaged in the cell that comprises the retroviral transcript. Also provided, in one embodiment, is a retroviral transcript that is obtainable by the method. In some embodiments, the retroviral transcript is obtained by the method.

### BRIEF DESCRIPTION OF THE DRAWINGS

FIG. 1 shows total p24 titers (nanogram per mL) measured in filter-harvested and concentrated (denoted by ‘10×’) LVV supernatants after single freeze-thaw of sample. A transduction LVV with known titer was used as Transduction Control.

FIG. 2 shows histogram of surface ddBCMA showing positive staining by MFI peaks (on right of solid line) of Jurkat cells transduced with pKT01 and pKT02 at 1:3125 viral dilution. Staining of BCMA-Dylight650 (rhBCMA mFc DL650; Lot: KPL-21-0148; 1.02 mg/mL at 0.1 mL; dilute 1:600 per 2.0×E5 cells) antigen was performed using untransduced and unstained Jurkats as control.

FIG. 3 shows Jurkat CAR expression titers (transduced units per mL) of pKT01 and pKT02. Both vectors were concentrated 10-fold and diluted in Jurkats for transduction. Transfection and transduction LVV with known titers were used as Transfection and Transduction Controls, respectively.

FIG. 4 shows Jurkat VCN titers for pKT01 and pKT02. The titers were calculated by taking an average of transduced viral dilutions 1:625 and 1:3125 in assay. Transfection and transduction LVV with known titers were used as Transfection and Transduction Controls, respectively.

FIG. 5 illustrates the configuration of transfer plasmids pKT01 and pKT02.

### DETAILED DESCRIPTION OF THE DISCLOSURE

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, an antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, a, B, y, and 8, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation and/or apheresis. Additional methods of isolating immune cells for an immune cell therapy are disclosed in U.S. Patent Publication No. 2013/0287748, which is herein incorporated by references in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, an RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA, RNA, or other genetic segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

### Transfer Vectors

Cell therapies and gene therapies are associated with high costs, and a significant factor contributing to this is the complexity and expense of the manufacturing process. Retroviral vectors are commonly used in cell therapies given their ease of manipulation, stable integration into the target genome, and a wide range of target cells. Retroviral vectors can be prepared in cells enclosing a transfer plasmid. The term “transfer plasmid” as used herein refers to a plasmid that encodes a viral transcript with an appropriate promoter driving the production of the viral transcript.

An important step in the manufacturing of retroviral vectors is the transcription from a transfer plasmid which is then exported to the cytoplasm for packaging. The transcription of both the viral and therapeutic protein transcript is typically driven by the LTR. In the retroviral vector, a eukaryotic promoter is used to drive the expression of the therapeutic protein to be produced in the target cell. The elongation factor 1-alpha 1 (EF1α) promoter is a promoter that can be used for this purpose. The EF1α promoter includes a core region (SEQ ID NO:2) and an intron (SEQ ID NO: 3). An EF1α promoter that includes both the promoter core and the intron is also referred to as a full-length EF1α promoter.

As demonstrated in the accompanying experimental examples, a transfer plasmid, designated pKT01, was designed to include a lentiviral vector (LVV) transcript that contains a CAR (ddBCMA CAR) under the regulation of a full-length EF1α promoter, along with an HIV Rev response element (RRE) (SEQ ID NO:1). Such a transfer plasmid exhibited excellent LVV production efficiency. Most of the packaged LVV genomes retained the EF1α intron due to the presence of the RRE and co-expression of the Rev protein. Retention of the EF1α intron in the final LVV genome, however, could in some instances lead to undesirable high expression, and hence potential adverse effects, of the CAR in the target cells.

In the next round of design, the EF1α intron was removed from the full-length EF1α promoter, in plasmid pKT02, which retained the high transcription, nuclear export and packaging efficiency of pKT01. Yet, the packaged LVV from pKT02 had moderately lower protein expression as compared to those from pKT01. pKT02, therefore, could present an excellent choice for clinical development or otherwise provide an alternative for selecting or modulating the level of expression of the CAR.

In accordance with one embodiment of the present disclosure, therefore, provided is a DNA molecule (e.g., a plasmid) that encodes a retroviral transcript. In accordance with another embodiment of the present disclosure, provided is a DNA molecule (e.g., a plasmid) which, when transcribed in a cell such as a eukaryotic cell, generates a retroviral transcript. In some embodiments, the retroviral transcript includes a transgene and an elongation factor 1-alpha 1 (EF1α) promoter operably linked to the transgene. In some embodiments, the EF1α promoter does not include an EF1α intron.

As shown in Example 1, when the DNA molecule included a full EF1α promoter, the majority of the produced transcripts retained the EF1α intron of the promoter. When the DNA molecule of the instant disclosure is used, no produced retroviral transcript would include the EF1α intron. In some embodiments, therefore, provided is a DNA molecule which, when transcribed in a eukaryotic cell, generates a plurality of retroviral transcripts, wherein each retroviral transcript comprises (a) a transgene and (b) an elongation factor 1-alpha 1 (EF1α) promoter operably linked to the transgene, and wherein less than 90% of the retroviral transcripts further comprise an EF1α intron.

In some embodiments, less than 80% of the retroviral transcripts further comprise an EF1α intron. In some embodiments, less than 70%, 60%, 50%, 40%, 30%, 20%, 15%, 10%, 5%, 2%, 1%, 0.5%, of 0.1% of the retroviral transcripts further comprise an EF1α intron. In some embodiments, none of the retroviral transcripts include an EF1α intron.

In some embodiments, the eukaryotic cell is a mammalian cell. In some embodiments, the eukaryotic cell is a human cell.

In some embodiments, each retroviral transcript further comprises: (c) two long terminal repeats (LTR) flanking the EF1α promoter and the transgene; and (d) a Rev response element (RRE). The EF1α promoter without the intron, also referred to herein as the EF1α promoter core, is configured to drive the expression of the transgene in a eukaryotic cell. An example sequence for the EF1α promoter core is provided in SEQ ID NO:2. In some embodiments, the EF1α promoter core has a sequence that has at least 70%, 75%, 80%, 85%, 90%, 95%, 96%, 97%, 98%, or 99% sequence identity to SEQ ID NO:2.

In some embodiments, the EF1α promoter core is not immediately followed by the intron (SEQ ID NO:3) or a sequence having at least 70%, 75%, 80%, 85%, 90%, 95%, 96%, 97%, 98%, or 99% sequence identity to SEQ ID NO:3. In some embodiments, the retroviral transcript in the DNA molecule, at any location between the EF1α promoter core and the transgene, does not include the intron (SEQ ID NO:3) or a sequence having at least 70%, 75%, 80%, 85%, 90%, 95%, 96%, 97%, 98%, or 99% sequence identity to SEQ ID NO:3. In some embodiments, the retroviral transcript in the DNA molecule, at any location within the retroviral transcript, does not include the intron (SEQ ID NO:3) or a sequence having at least 70%, 75%, 80%, 85%, 90%, 95%, 96%, 97%, 98%, or 99% sequence identity to SEQ ID NO:3. In some embodiments, the DNA molecule, at any location within the DNA molecule, does not include the intron (SEQ ID NO:3) or a sequence having at least 70%, 75%, 80%, 85%, 90%, 95%, 96%, 97%, 98%, or 99% sequence identity to SEQ ID NO:3.

In some embodiments, the retroviral transcript further includes a Rev response element (RRE). The Rev response element (RRE) is a highly structured, ˜350 nucleotide RNA segment present in the Env coding region of unspliced and partially spliced viral mRNAs. In the presence of the HIV-1 accessory protein Rev, mRNAs that contain the RRE can be exported from the nucleus to the cytoplasm for downstream events such as translation and virion packaging.

The RRE is a highly structured RNA element. Computational analysis and chemical and enzymatic probing indicate that an RRE contains multiple stem loops and bulges. Rev binds to RRE in a sequence specific manner with Rev-RNA recognition mediated by a 17-residue a-helical stretch on Rev, the Arginine-Rich-Motif (ARM). Images of the tertiary structure of RRE (and the Rev-RRE complex) show a globular “head” with a long stalk extending from it.

In addition to the HIV-1, HIV-2 and SIV (Simian Immunodeficiency Virus) also have Rev-RRE systems. Moreover, some betaretroviruses use a Rem/RmRE system, and deltaretroviruses use Rex/RxRRE systems. The RmRE and RxRRE elements, it is appreciated, are also encompassed by the term of RRE as used herein.

An example HIV-1 RRE sequence is provided in SEQ ID NO:1. Examples of of the HIV-1 RRE include those with GenBank accession Nos. KF586402.1, KF586403.1, KF586404.1, KF586405.1, KF586406.1, KF586407.1, KF586408.1, KF586409.1, KF586410.1, KF586411.1, KF586412.1, KF586413.1, KF586414.1, KF586415.1, KF586416.1, KF586417.1, KF586418.1, KF586419.1, KF586420.1, and KT368137.1, without limitation. An example RRE from HIV-2 is provided in GenBank accession No. KY272752.1 (nucleotides 10949 to 11164, SEQ ID NO:6). In some embodiments, the RRE is one of an HIV subtype selected from the group of subtypes A1, A2, B, C, D, F1, F2, G and H.

In some embodiments, the RRE includes the sequence of SEQ ID NO: 1 or 6, or a sequence having at least 70%, 75%, 80%, 85%, 90%, 95%, 96%, 97%, 98%, or 99% sequence identity to SEQ ID NO:1 or 6. In some embodiments, the retroviral transcript further includes a sequence that encode a corresponding Rev protein. In some embodiments, the RRE is disposed upstream of the EF1α promoter core. In some embodiments, the RRE is disposed downstream of the transgene.

TABLE A
Sequences

| | | SEQ ID |
|---|---|---|
| Name | Sequence | NO: |
| RRE | AGTAGCACCCACCAAGGCAAAGAGAAGAGTGGTGCAGAGAGAAAAAAGAG | 1 |
| | CAGTGGGAATAGGAGCTTTGTTCCTTGGGTTCTTGGGAGCAGCAGGAAGC | |
| | ACTATGGGCGCAGCGTCAATGACGCTGACGGTACAGGCCAGACAATTATT | |
| | GTCTGGTATAGTGCAGCAGCAGAACAATTTGCTGAGGGCTATTGAGGCGC | |
| | AACAGCATCTGTTGCAACTCACAGTCTGGGGCATCAAGCAGCTCCAGGCA | |
| | AGAATCCTGGCTGTGGAAAGATACCTAAAGGATCAACAGCTCCTGGGGAT | |
| | TTGGGGTTGCTCTGGAAAACTCATTTGCACCACTGCTGTGCCTTGGAATG | |
| | CTAGT | |
| EF1α | CGTGAGGCTCCGGTGCCCGTCAGTGGGCAGAGCGCACATCGCCCACAGTC | 2 |
| promoter | CCCGAGAAGTTGGGGGGAGGGGTCGGCAATTGAACCGGTGCCTAGAGAAG | |
| core | GTGGCGCGGGGTAAACTGGGAAAGTGATGTCGTGTACTGGCTCCGCCTTT | |
| | TTCCCGAGGGTGGGGGAGAACCGTATATAAGTGCAGTAGTCGCCGTGAAC | |
| | GTTCTTTTTCGCAACGGGTTTGCCGCCAGAACACAGGTGTCGTGA | |
| EF1α | AGAACACAGGTAAGTGCCGTGTGTGGTTCCCGCGGGCCTGGCCTCTTTAC | 3 |
| intron | GGGTTATGGCCCTTGCGTGCCTTGAATTACTTCCACCTGGCTGCAGTACG | |
| | TGATTCTTGATCCCGAGCTTCGGGTTGGAAGTGGGTGGGAGAGTTCGAGG | |
| | CCTTGCGCTTAAGGAGCCCCTTCGCCTCGTGCTTGAGTTGAGGCCTGGCC | |
| | TGGGCGCTGGGGCCGCCGCGTGCGAATCTGGTGGCACCTTCGCGCCTGTC | |
| | TCGCTGCTTTCGATAAGTCTCTAGCCATTTAAAATTTTTGATGACCTGCT | |
| | GCGACGCTTTTTTTCTGGCAAGATAGTCTTGTAAATGCGGGCCAAGATCT | |
| | GCACACTGGTATTTCGGTTTTTGGGGCCGCGGGCGGCGACGGGGCCCGTG | |
| | CGTCCCAGCGCACATGTTCGGCGAGGCGGGGCCTGCGAGCGCGGCCACCG | |
| | AGAATCGGACGGGGGTAGTCTCAAGCTGGCCGGCCTGCTCTGGTGCCTGG | |
| | CCTCGCGCCGCCGTGTATCGCCCCGCCCTGGGCGGCAAGGCTGGCCCGGT | |
| | CGGCACCAGTTGCGTGAGCGGAAAGATGGCCGCTTCCCGGCCCTGCTGCA | |
| | GGGAGCTCAAAATGGAGGACGCGGCGCTCGGGAGAGCGGGCGGGTGAGTC | |
| | ACCCACACAAAGGAAAAGGGCCTTTCCGTCCTCAGCCGTCGCTTCATGTG | |
| | ACTCCACTGAGTACCGGGCGCCGTCCAGGCACCTCGATTAGTTCTCGTGC | |
| | TTTTGGAGTACGTCGTCTTTAGGTTGGGGGGAGGGGTTTTATGCGATGGA | |
| | GTTTCCCCACACTGAGTGGGTGGAGACTGAAGTTAGGCCAGCTTGGCACT | |
| | TGATGTAATTCTCCTTGGAATTTGCCCTTTTTGAGTTTGGATCTTGGTTC | |
| | ATTCTCAAGCCTCAGACAGTGGTTCAAAGTTTTTTTCTTCCATTT | |
| BCMA | MGSWSEFWARLGAIRERLDALGGSEAELAAFEKEIAAFESELQAYKGKGN | 4 |
| binder | PEVEKLRYTAGTIKRFLQAYRHN | |
| BCMA- | MGSWSEFWARLGAIRERLDALGGSEAELAAFEKEIAAFESELQAYKGKGN | 5 |
| CAR | PEVEKLRYTAGTIKRFLQAYRHNGGGGDGGGGSGTTTPAPRPPTPAPTIA | |
| | SQPLSLRPEACRPAAGGAVHTRGLDFACDIYIWAPLAGTCGVLLLSLVIT | |
| | LYCKRGRKKLLYIFKQPFMRPVQTTQEEDGCSCRFPEEEEGGCELRVKES | |
| | RSADAPAYKQGQNQLYNELNLGRREEYDVLDKRRGRDPEMGGKPRRKNPQ | |
| | EGLYNELQKDKMAEAYSEIGMKGERRRGKGHDGLYQGLSTATKDTYDALH | |
| | MQALPPR | |
| HIV-2 | GGTTCTTGGGTTTTCTCGCAACAGCAGGTTCTGCAATGGGCGCGGCGTCC | 6 |
| RRE | CTGACGCTGTCGGCTCAGTCCCGGACTTTACTGGCCGGGATAGTGCAGCA | |
| | ACAGCAACAGCTGTTGGACGTGGTCAAGAGACAACAAGAACTGTTGCGAC | |
| | TGACCGTCTGGGGAACGAAAAACCTCCAGGCAAGAGTCACTGCTATAGAG | |
| | AAGTACCTACAGGACC | |

The retroviral transcript (or retroviral vector) may be any retroviral transcript or vector known in the art. In one embodiment, the retroviral transcript is a transcript of a Moloney Murine Leukemia Virus (MoMLV), a gammaretrovirus, or Murine Stem Cell Virus (MSCV), which are commonly used for stable gene expression in dividing cells.

In some embodiments, the retroviral transcript is a lentiviral transcript, which is derived from the HIV virus. When a host cell is infected with a lentivirus, the genomic RNA is reverse transcribed into a DNA intermediate which is integrated into the chromosomal DNA of infected cells.

In some embodiments, the retroviral transcript further includes elements useful for the production and/or expression of the retroviral transcript. In one embodiment, the retroviral transcript includes one or more of the gag, the pol, and the env gene. In some embodiments, the gag gene encodes the internal structural (matrix, capsid, and nucleocapsid) proteins. In some embodiments, the pol gene encodes the RNA-directed DNA polymerase (reverse transcriptase). In some embodiments, the env gene encodes viral envelope glycoproteins.

In some embodiments, the retroviral transcript includes one or more genes such as vit, vpr, tat, rev, vpu, nef, and vpx. In some embodiments, the retroviral transcript further includes a posttranscriptional regulatory element (PRE). A non-limiting example is the Woodchuck posttranscriptional regulatory element (WPRE).

In some embodiments, these elements are flanked by two long terminal repeat (LTR) sequences. The flanking LTRs (or the 5′ and 3′ LTRs) serve to promote transcription and polyadenylation of the virion RNAs. The LTRs contain cis-acting sequences useful for viral replication.

Adjacent to the 5′ LTR are sequences useful for reverse transcription of the genome (e.g., tRNA primer binding site) and for efficient encapsidation of viral RNA into particles (e.g., the Psi site, ψ).

In some embodiments, the retroviral transcript further includes a viral promoter for driving the transcription of the retroviral transcript. In some embodiments, the viral promoter is a Cytomegalovirus (CMV) promoter. In some embodiments, the viral promoter is a SV40 promoter. In some embodiments, the viral promoter is a Rous Sarcoma Virus (RSV) promoter.

### Transgene

The transgene of the retroviral transcript can be any gene of interest (GOI), such as one encoding a protein useful for treating a disease or condition in a subject (a “therapeutic protein”), such as a human patient.

Non-limiting examples of proteins useful for treating diseases or conditions include insulin (for treating diabetes), phenylalanine hydroxylase (for treating PKU (Phenylketonuria) disease), erythropoietin (for treating anemia), filgrastim (G-CSF) (for treating neutropenia), interferons (for treating cancer, multiple sclerosis, and viral infections), tumor necrosis factor (TNF) inhibitors (for treating autoimmune diseases, such as rheumatoid arthritis and Crohn's disease), monoclonal antibodies (for treating cancer, autoimmune diseases, and some viral infections), follitropin alfa (for treating infertility), coagulation factors (for treating bleeding disorders, such as hemophilia), enzyme replacement therapy enzymes (for treating lysosomal storage disorders, such as Gaucher's disease), an growth hormones (for treating growth hormone deficiency).

Yet another important example of a protein encoded by the transgene is a chimeric antigen receptor (CAR) or T-cell receptor (TCR). Both CAR and TCR include an antigen-binding portion, which is typically an antibody or antigen-binding fragment of an antibody. However, other antigen-binding peptides can also be used, such as D domains.

A D domain (Dimerization domain) can be a natural protein domain found in the upstream of the F-box domain, which is a conserved dimerization motif located in WD40 repeat F box proteins, such as Cdc4, Met30, β-TrCP and Pop1/2.

A D domain is comprised of three alpha helices which generate a parallel dimer by self-associating in a right-handed super-helical way. There are two possible configurations for this domain's N terminus, an unstructured loop and an amphipathic alpha-helix (H0). Interactions with the adjacent thyroid hormone receptor ligand-binding domain's (TR-LBD), AF-2 coactivator-binding groove are important for the creation of the H0 structure of D-domain.

Artificial D domains can be screened from D domain libraries, such as those disclosed in WO2019099433A2, including ones that specifically bind BCMA, CD123, AFP, AFP p26, CS1, and HER2. In one embodiment, the D domain having specificity to BCMA has the amino acid sequence of SEQ ID NO:4, or an amino acid sequence having at least 70%, 75%, 80%, 85%, 90%, 95%, 96%, 97%, 98%, or 99% sequence identity to SEQ ID NO:4.

In addition to BCMA, the antigen-binding portion of the CAR or TCR can have specificity to one or more of 5T4, alphafetoprotein, CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, or a combination thereof.

In some embodiments, the antigen-binding portion of the CAR or TCR can have specificity to 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Ab1), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECLI), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYPIB1), DNAM-1 (CD226), desmoglein 4, DR3, DR5, E-cadherin neoepitope, epidermal growth factor receptor (EGFR), EGFIR, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor «, Folate receptor β, Fos-related antigen 1, Fucosyl, Fucosyl GM1: GM2. ganglioside G2 (GD2). ganglioside GD3 (aNeu5Ac (2-8) aNeu5Ac (2-3) bDGalp (1-4) bDGlcp (1-1) Cer),o-acetyl-GD2 ganglioside (OAcGD2), GITR (TNFRSF 18), GM1, ganglioside GM3 (aNeu5Ac (2-3) bDGalp (1-4) bDGlcp (1-1) Cer), GP 100, hexasaccharide portion of globoH glycoceramide (GloboH), glycoprotein 75, Glypican-3 (GPC3), glycoprotein 100 (gplOO), GPNMB, G protein-coupled receptor 20 (GPR20), G protein-coupled receptor class C group 5, member D (GPRC5D), Hepatitis A virus cellular receptor 1 (HAVCR), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5ß1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis (Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1): melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTI, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUCI), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA,NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PRI), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEMI/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member IA (XAGEI).

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAMI), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3 DPI), CD158D (KIRDL4), CD158F1 (KIR2DL5A), CD158F2 (KIR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRFI), IL-2R beta, IL-2R gamma, IL-7R alpha, LFA-1, SLAMF9, LAT, GADS (GrpL), SLP-76 (LCP2), PAGI/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAMI, CRT AM, cytokine receptor, DAP-10, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRFI), OX-40, PAG/Cbp, programmed death-1 (PD-1), PSGLI, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAMI, CRT AM, cytokine receptor, DAP-10, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRFI), OX-40, PAG/Cbp, programmed death-1 (PD-1), PSGLI, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40(CD134), CD30, CD40, PD-1, ICOS(CD278), lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-1BB costimulatory domain. In certain instances, a CAR includes a CD28 costimulatory domain. In certain instances, a CAR includes a DAP-10 costimulatory domain.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d. In some embodiments, the ITAM includes CD3 zeta.

In some embodiments, the CAR includes the amino acid sequence of SEQ ID NO:5, or an amino acid sequence having at least 70%, 75%, 80%, 85%, 90%, 95%, 96%, 97%, 98%, or 99% sequence identity to SEQ ID NO:5.

In some embodiments, the transgene is at least 500 nucleotides in length. In some embodiments, the transgene is at least 600, 700, 800, 900, 1,000, 1,500, 2,000, 3,000, 4,000, 5,000 6,000, 7,000, 8,000, 9,000, 10,000, 11,000, 12,000, 13,000, 14,000, 15,000 or 20,000 nucleotides in length. In some embodiments, the transgene is not longer than 10,000, 11,000, 12,000, 13,000, 14,000, 15,000, 20,000, 25,000 or 30,000 nucleotides in length.

### Preparation of Retroviral Transcripts and Transduced Cells

The present disclosure, in some embodiments, further provides methods of using the DNA molecule (e.g., transfer plasmid) to prepare retroviral transcripts and vectors, transcripts and vectors prepared by such methods, as well as cells transduced or transfected with such vectors. Also provided are methods of using the vectors or cells for treating a disease or condition in a subject in need thereof.

In one embodiment, a method is provided for preparing a retroviral transcript, which method entails introducing a DNA molecule of the present disclosure to a eukaryotic cell and then culturing the cell to allow the DNA molecule to transcribe to generate the retroviral transcript. The eukaryotic cell may be a primary cell or a cell line known in the art, such as HEK293T cells. Also provided are the produced retroviral transcripts, preferably packaged retroviral transcripts. Still further provided are cells transfected with or containing the DNA molecule.

To prepare a cell that encloses one or more of such retroviral transcripts, the process can include (1) acquiring and/or enriching a population of lymphocytes (e.g., T cells) obtained from a donor subject; (2) transducing the population of lymphocytes with a retroviral transcript of the present disclosure; and (3) harvesting transduced lymphocytes. In some embodiments, the harvested lymphocytes are administered to a patient, such as the donor. In some embodiments, the harvested lymphocytes are cryopreserved.

In some embodiments, at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% of lymphocytes in the harvested sample are naïve cells. In some embodiments, at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% of T cells in the harvested sample are naïve T cells.

In some embodiments, a naïve T cell is characterized with one or more markers such as CD45RA⁺, CCR7⁺, CD62L⁺, CD27⁺, CD28⁺, CD127⁺, CD132⁺, CD25⁻, CD44⁻, CD45RO⁻, and HLA-DR. In one embodiment, a naïve T cell is characterized with CD45RA⁺ and CCR7⁺. In one embodiment, a naïve T cell is characterized with CD45RA⁺, CCR7⁺, CD62L+, CD27⁺, and CD28⁺. In one embodiment, a naïve T cell is characterized additionally with CD127⁺, CD132⁺, CD25⁻, CD44⁻, CD45RO⁻, and/or HLA-DR″.

### Compositions and Treatments

The cells, e.g., allogeneic or autologous cells transduced with the retroviral vectors, of the present disclosure can be used for treating various diseases and conditions, in particular cancer. In one embodiment, the cancer may comprise Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, multiple myeloma, acute lymphoblastic leukemia, acute myelogenous leukemia, chronic lymphocytic leukemia, chronic myelogenous leukemia, lymphoma, Hodgkin's lymphoma, non-Hodgkin's lymphoma, and urinary bladder cancer.

Another embodiment described herein is a method of treating a cancer in a subject in need thereof comprising administering an effective amount, e.g., therapeutically effective amount of a composition comprising a transfected cell of the present disclosure. Also provided are such compositions that include transfected lymphocytes disclosed herein and pharmaceutically acceptable excipients.

The quantity and frequency of administration will be determined by such factors as the condition of the patient, and the type and severity of the patient's disease, although appropriate dosages may be determined by clinical trials. In some embodiments, the cancer is characterized with the expression of an antigen targeted by the CAR or TCR molecule, such as BCMA.

In other embodiments, methods comprising administering a therapeutically effective amount of transduced T cells contemplated herein or a composition comprising the same, to a patient in need thereof, alone or in combination with one or more therapeutic agents, are provided. In certain embodiments, the cells of the disclosure are used in the treatment of patients at risk for developing a cancer. Thus, the present disclosure provides methods for the treatment or prevention of a cancer comprising administering to a subject in need thereof, a therapeutically effective amount of the modified T cells of the disclosure.

One of ordinary skill in the art would recognize that multiple administrations of the compositions of the disclosure may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

The methods for administering the cell compositions described herein includes any method which is effective to result in reintroduction of ex vivo genetically modified immune effector cells that either directly express an TCR or CAR in the subject or on reintroduction of the genetically modified progenitors of immune effector cells that on introduction into a subject differentiate into mature immune effector cells that express the TCR or CAR. One method comprises transducing peripheral blood T cells ex vivo with a nucleic acid construct in accordance with the present disclosure and returning the transduced cells into the subject.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### EXAMPLES

### Example 1. Quantitation for ddBCMA CAR Lentiviral Vectors pKT01 and pKT02

This example conducted quantitation analysis for two lentiviral vector (LVV)-encoding transfer plasmids, pKT01 and pKT02, for expressing a chimeric antigen receptor (CAR) referred to as ddBCMA (ddBCMA CAR). The transduction efficiency and expression of the two vectors were determined.

The ddBCMA CAR (SEQ ID NO:5) includes a D Domain having binding specificity to the human BCMA protein (SEQ ID NO:4), linked to a CD8a hinge, a CD8a transmembrane domain, a 4-1BB costimulatory domain and a CD3ζ stimulatory domain.

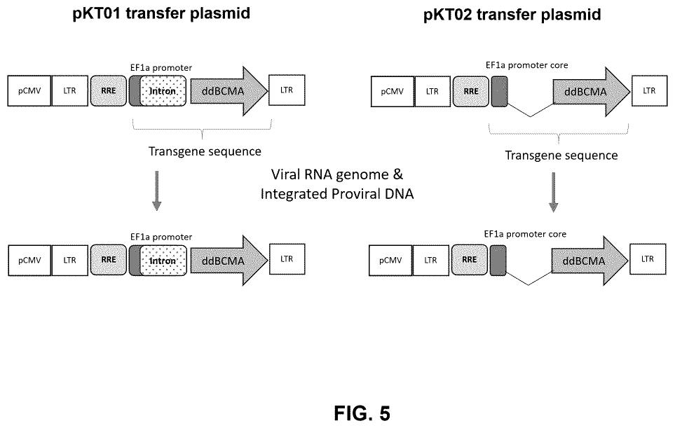

The pKT01 LVV-encoding transfer plasmid (illustrated in FIG. 5) includes a Cytomegalovirus (CMV) promoter driving the transcription of the LVV retroviral genome. In pKT01, the LVV transcript includes the coding sequence of ddBCMA CAR preceded with an EF1α elongation factor 1-alpha 1 (EF1a) promoter operably linked to the ddBCMA CAR. Here, the EF1α promoter includes an EF1α intron (SEQ ID NO:3). In addition, the LVV transcript includes an HIV Rev response element (RRE) (SEQ ID NO:1) upstream of the EF1α promoter and a Woodchuck posttranscriptional regulatory element (WPRE) downstream of the ddBCMA CAR. All of these sequences, except the CMV promoter, are flanked by two long terminal repeats (LTR).

Compared to the pKT01 transfer plasmid, the pKT02 transfer plasmid (FIG. 5) does not include the EF1α intron (also referred to as the “EF1α promoter core”).

### Lentiviral Vector Production

Lentiviral vector (LVV) production was performed according to the standard operating procedure. LVV were produced using suspension HEK293T cells in small-scale transient transfections. Transfection was performed by diluting packaging plasmids with polyethylenimine (PEI) in OPTIMEM® media. A final concentration of 2 μg/mL DNA, transfer vector and packaging plasmids combined, was used. After 48 hours of incubation, cells released viral particles which were harvested by filtration. The LVV were further concentrated 10-fold using Lenti-X concentration buffer to increase the total viral product. These cell-free supernatants were the final product used to transduce primary T-cells for downstream experiments.

### Total p24 Titer

The p24 antigen is a capsid protein found on HIV-I based vectors and was used to measure the total viral yield of LVV. An automated ELISA assay was used to measure total p24 in crude supernatant. Final titers (ng/mL) were calculated by converting the raw triplicate readouts from picogram/mL to nanogram/mL.

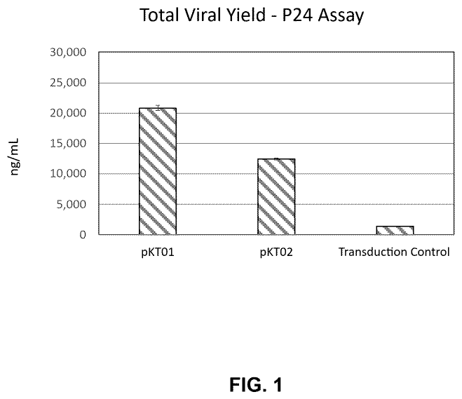

Results from the assay (FIG. 1) show that pKT02 produced 12458 ng/mL p24, and pKT01 produced 20824 ng/mL p24. The p24 titer inferring total viral yield was lower by 40% for pKT02. The viral yield was in linear range of a typical high-titer virus.

### Jurkat CAR Expression Titer

LVV supernatants were serially diluted 5-fold and transduced in Jurkat E6-1 cell line. Surface expression of the ddBCMA CAR was measured on Day 4 after transduction. Quantifying expression titer allows to determine the effective transgene expression in a pool of transduced T-cells. Samples were stained with viability dye followed by a ddBCMA CAR antibody bound to conjugate fluorophore Dylight 650. Samples were processed in Attune flow cytometer to detect CAR positive population.

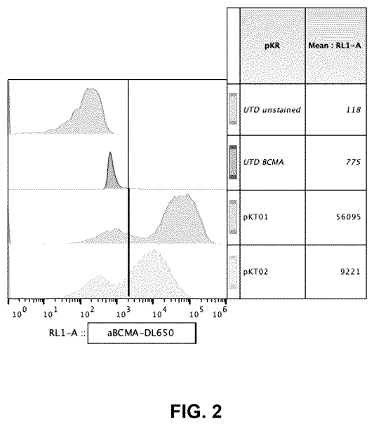

The histograms in FIG. 2 show CAR expression of viral dilution 1:3125 in transduced samples. The mean fluorescence intensity (MFI) was 56095 for pKT01 and 9221 for pKT02. The % CAR positive population was 88.9% for pKT01 and 69% for pKT02. The specific readouts used to determine final expression titer are included in Table 1.

TABLE 1
Expression Readouts

| | Viral | | |
|---|---|---|---|
| | Dilution | pKT01-10x | pKT02-10x |
| MFI | 625 | 195928 | 54133 |
| | 3125 | 56095 | 9221 |
| CAR Positive (%) | 625 | 99.6 | 97.5 |
| | 3125 | 88.9 | 69 |
| Expression Titer | 625 | 1.25E+08 | 1.22E+08 |
| (TU/mL) | 3125 | 5.56E+08 | 4.31E+08 |
| Final Average | | 3.40E+08 | 2.77E+08 |
| Expression Titer | | | |
| (TU/mL) | | | |

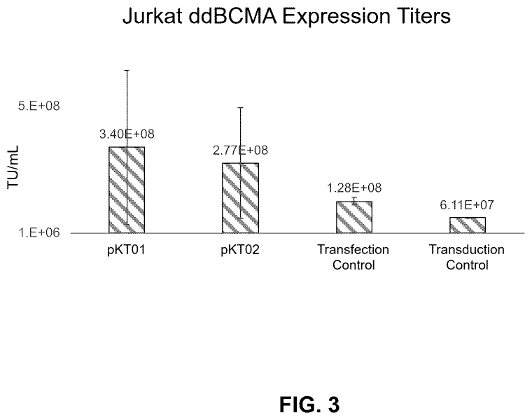

The expression titers (FIG. 3) were calculated by taking the average of % CAR measured in viral dilutions 1:625 and 1:3125 and were normalized to Day 4 cell concentration and seeded cell volume. MFI and correlating expression titers showed reduced CAR expression by about 19% in pKT02 compared to pKT01. The MFI, % positive CAR, and correlating expression titers were all within linear range of a typical high-titer lentiviral vector.

### Jurkat VCN Titer

LVV supernatants were diluted and transduced in Jurkat E6-1 cell line. Vector copy numbers were measured on Day 7 after transduction. The droplet digital PCR assay enabled estimation of the average numbers of reverse transcribed LVV vector DNA molecules per cell, or vector copy number (VCN), in a DNA sample from transduced cells. A crude lysate was prepared in Quick DNA Extract buffer. Lysates were incubated in a reaction mix containing primers and probes for LVV (i.e., FAM-SIN18) and for a reference target (i.e., HEX-UC378) in genomic DNA, Dral restriction enzyme, template DNA, and water. The final titer (TU/mL) was calculated using raw CNV, transduced cell concentration, viral dilution, and cell volume.

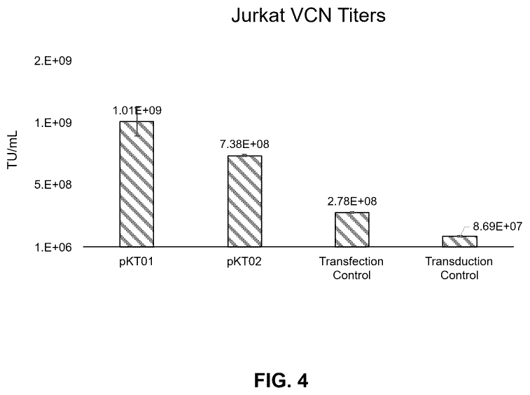

The results (FIG. 4) show that pKT01 had a VCN titer of 1.01e9 TU/mL and pKT02 had a VCN titer of 7.38e8 TU/mL. The results were closely comparable between both LVV, which shows that they have similar integration efficiencies in host cells. The VCN titer inferring transduction efficiency was lower by 27% in pKT02. The titer values achieved by both vectors were in linear range of expected copy number for a concentrated vector. These VCN titers were used to determine MOI for transducing primary and donor T-cells in downstream in-vitro experiments.

This example measured the p24 titer, transduction efficiency, and surface CAR expression of pKT02 (with the EF1α promoter core) driving ddBCMA CAR and pKT01 (with full-length EF1α promoter) driving ddBCMA CAR. Both pKT01 and pKT02 constructs produced high-titer virus with high transduction efficiency in T-cells. pKT02 vector exhibited ˜19% reduced surface CAR expression as compared to pKT01.

The results reported here suggest that, in the pKT01 plasmid, inclusion of the RRE led to superior efficiency in nuclear export and LVV packaging. Most of the transcripts (as well as the reverse-transcribed and integrated DNA) retained the EF1α intron (FIG. 5). The retained EF1α intron led to high expression of the ddBCMA CAR in transduced cells. Such high expression, however, could be associated with increased risk of adverse effects.

In the next generation of design, the pKT02 plasmid did not include the EF1α promoter intron. This plasmid, as shown above, had high production and packaging efficiency and yet the resulting transduced cells had desired, slightly reduced, expression levels for the ddBCMA CAR. The pKT02 plasmid provides an excellent or desirable transfer plasmid for efficient manufacturing of packaged LVV which can be used to express an optimal amount the ddBCMA CAR in the target cell.

The pKT02 plasmid may also have advantages when compared to other plasmids that lack both the EF1α promoter intron as well as the RRE element. These advantages include, for example, increased number of viral particles, increased purity with fewer inactive, empty particles, and improved performance with increased expression and integration titer.

While a number of embodiments have been described, it is apparent that the disclosure and examples may provide other embodiments that utilize or are encompassed by the compositions and methods described herein. Therefore, it will be appreciated that the scope of the invention is to be defined by that which may be understood from the disclosure and the appended claims rather than by the embodiments that have been represented by way of example.

1. A DNA molecule encoding a retroviral transcript, wherein the retroviral transcript comprises:
(a) a transgene;
(b) an elongation factor 1-alpha 1 (EF1α) promoter operably linked to the transgene;
(c) two long terminal repeats (LTR) flanking the EF1α promoter and the transgene; and
(d) a Rev response element (RRE),
wherein the DNA molecule further comprises a viral promoter for driving the transcription of the retroviral transcript, and wherein the EF1α promoter does not include an EF1α intron.

2. The DNA molecule of claim 1, wherein the retroviral transcript is a lentiviral transcript.

3. The DNA molecule of claim 1, wherein the viral promoter is a Cytomegalovirus (CMV) promoter, a SV40 promoter, or a Rous Sarcoma Virus (RSV) promoter.

4. The DNA molecule of claim 1, wherein the RRE is upstream to the EF1α promoter or downstream to the transgene.

5. The DNA molecule of claim 1, wherein the RRE is a human immunodeficiency viruses (HIV) RRE.

6. The DNA molecule of claim 5, wherein the RRE is an RRE of an HIV subtype selected from the group of subtypes A1, A2, B, C, D, F1, F2, G and H.

7. The DNA molecule of claim 1, wherein the RRE comprises a nucleic acid sequence having at least 90% sequence identity to SEQ ID NO:1.

8. The DNA molecule of claim 1, wherein the EF1α promoter comprises a nucleic acid sequence having at least 90% sequence identity to SEQ ID NO:2.

9. The DNA molecule of claim 1, wherein the intron of the EF1α promoter comprises a nucleic acid having at least 90% sequence identity to SEQ ID NO:3.

10. The DNA molecule of claim 1, wherein the transgene is a therapeutic protein.

11. The DNA molecule of claim 10, wherein the therapeutic protein comprises chimeric antigen receptor (CAR).

12. The DNA molecule of claim 11, wherein the CAR comprises an antigen-binding peptide.

13. The DNA molecule of claim 12, wherein the antigen-binding peptide has binding specificity to the B-cell maturation antigen (BCMA).

14. The DNA molecule of claim 13, wherein the antigen-binding peptide comprises an amino acid sequence having at least 90% to SEQ ID NO:4.

15. The DNA molecule of claim 11, wherein the CAR further comprises one or more of a signal peptide, a transmembrane domain, a costimulatory domain from 4-1BB, CD28, ICOS(CD278), OX40(CD134), or DAP-10, and a CD3ζ stimulatory domain.

16. The DNA molecule of claim 15, wherein the CAR comprise an amino acid sequence having at least 90% sequence identity to SEQ ID NO:4.

17. The DNA molecule of claim 1, wherein the retroviral transcript further comprises a posttranscriptional regulatory element (PRE).

18. The DNA molecule of claim 17, wherein the PRE is a Woodchuck PRE (WPRE).

19. The DNA molecule of claim 1, wherein the DNA molecule is a plasmid.

20. A retroviral transcript encoded by the DNA molecule of claim 1.

21. An in vitro or in vivo cell comprising the DNA molecule of claim 1.

22. A method for preparing a retroviral transcript, comprising introducing a DNA molecule of claim 1 to a eukaryotic cell and culturing the cell to allow the DNA molecule to transcribe to generate the retroviral transcript.

23. A retroviral transcript, wherein the retroviral transcript is prepared by a method comprising introducing a DNA molecule of claim 1 to a eukaryotic cell and culturing the cell to allow the DNA molecule to transcribe to generate the retroviral transcript.

24. (canceled)

25. A retroviral transcript that is obtainable by the method of claim 22.

26. (canceled)

27. A DNA molecule which, when transcribed in a eukaryotic cell, generates a plurality of retroviral transcripts, wherein each retroviral transcript comprises:
(a) a transgene; and
(b) an elongation factor 1-alpha 1 (EF1α) promoter operably linked to the transgene;
wherein less than 80% of the retroviral transcripts further comprise an EF1α intron.

28-34. (canceled)

35. A method for preparing a retroviral transcript, comprising introducing a DNA molecule of claim 27 to a eukaryotic cell and culturing the cell to allow the DNA molecule to transcribe to generate the retroviral transcript.

36. A retroviral transcript that is obtainable by the method of claim 35.
